(19) United States
(12) Patent Application Publication
(10) Pub. No.: US 2023/0341387 A1
(43) Pub. Date: Oct. 26, 2023

(54) SPRING ELEMENT FOR ANALYZING AN ANALYTE

(71) Applicant: digid GmbH, Mainz (DE)

(72) Inventors: Frank FLACKE, Mainz (DE); Konstantin KLOPPSTECH, Mainz (DE); Constantin VON GERSDORFF, Mainz (DE); Malte BARTENWERFER, Mainz (DE); Nils KÖNNE, Mainz (DE)

(21) Appl. No.: 17/913,926

(22) PCT Filed: Mar. 23, 2021
(86) PCT No.: PCT/EP2021/057496

§ 371 Date: Sep. 23, 2022

(30) Foreign Application Priority Data
Mar. 23, 2020 (DE) 10 2020 107 918.4

(51) Int. Cl. G01N 33/543; G01N 33/563; G01N 33/569

(52) U.S. Cl. CPC G01N 33/5438; G01N 33/54346; G01N 33/563; G01N 33/56983; G01N 2333/165

(57) ABSTRACT

A spring element is provided for analysis of the presence of an analyte in a sample. The spring element includes a flexible main body having a conductivity detector zone and a binding zone. The conductivity of the conductivity detector zone is determined by electronic tunneling, ionization or hopping processes, and the conductivity detector zone is formed from nanoparticles embedded in a matrix that have higher electrical conductivity compared to the matrix material. Moreover, the binding zone includes at least one binding molecule that binds to the analyte and is coupled to the main body.

### CROSS REFERENCE TO RELATED APPLICATIONS

This application is a continuation of PCT Application No. PCT/EP2021/057496, filed Mar. 23, 2021, which claims priority to German Patent Application No. 10 2020 107 918.4, filed Mar. 23, 2020, the entire contents of each of which are hereby incorporated by reference in their entirety.

### TECHNICAL FIELD

The present invention relates to a spring element for analyzing an analyte. Specifically, the invention relates to miniaturized spring elements having a detector zone to which binding molecules that bind specifically to viral antigens are coupled, and to devices comprising these spring elements and to corresponding methods of detecting viruses.

### BACKGROUND

To date there are no available reliable and cost-effective point-of-care screening tests for real-time diagnosis of a SARS-CoV-2 virus infection at the point of treatment. The most commonly used test is based on a reverse transcriptase PCR method (Corman V M, Landt O, Kaiser M, et al. Detection of 2019 novel coronavirus (2019-nCoV) by real-time RT-PCR. Euro Surveill. 2020;25(3):2000045. doi:10.2807/1560-7917.ES.2020.25.3.2000045), which results in a relatively high workload by virtue of analysis in specialized diagnostic laboratories and an associated time demand of up to 3 days between sampling and availability of the result for medical personnel or the patient. This delay results both in a long period of uncertainty for the patient and in a significant delay both in the targeted treatment of the patient and in the application of suitable measures for controlling the epidemic.

Existing point-of-care test methods are based on the determination of the anti-viral immune reaction by means of measurement of IgG and IgM antibodies (Li Z, Yi Y, Luo X, et al. Development and Clinical Application of A Rapid IgM-IgG Combined Antibody Test for SARS-CoV-2 Infection Diagnosis [published online ahead of print, 2020 Feb. 27]. J Med Virol. 2020;10.1002/jmv.25727. doi:10.1002/jmv.25727) by means of lateral flow-based immunochromatography methods. However, virus-specific antibodies can be detected in the plasma only 7 to 10 days after infection. In the case of SARS-CoV-2, however, patients are already highly infectious in the first week after infection. This constitutes one of the main reasons for the rapid global spread of the COVID-19 pandemic.

WO 2007/088018 A1 proposes spring elements for use in biosensors, for example in DNA analysis. There is no disclosure of any application of the spring elements for detection of viruses.

### SUMMARY OF THE INVENTION

Against this background, it was the object of the present invention to overcome the disadvantages of the prior art that have been mentioned. More particularly, the invention was based on the object of providing a spring element for conversion of chemical and/or biochemical information from an analyte in a sample to an electrical signal and specifically of providing a device that enables PCR-independent detection of viruses.

This object is achieved by the spring element according to the claims and by the devices and methods according to the claims.

In a first aspect, the invention relates to a spring element for analyzing the presence of an analyte in a sample, comprising a flexible main body having a conductivity detector zone and a binding zone, wherein the electrical conductivity of the conductivity detector zone is determined by electronic tunneling, ionization or hopping processes, and wherein the conductivity detector zone is formed from nanoparticles embedded in a matrix that have higher electrical conductivity compared to the matrix material, and wherein the binding zone comprises at least one binding molecule that binds specifically to the analyte and is coupled to the main body.

In the present description, the singular and plural of the elements such as binding molecule/binding molecules, antigen/antigens etc. are generally used interchangeably.

The spring element described herein may be a miniaturized spring element.

Miniaturized spring elements having a flexible main body having a detector zone, the electrical conductivity (σ) of which is determined by electronic tunneling, ionization or hopping processes, and production thereof, are known to the person skilled in the art from WO 2007/088018 A1. Additionally knwon to the person skilled in the art from U.S. Pat. Nos. 4,426,768; 4,510,178 and 7,963,171 B2 are production processes for corresponding detector zones based on a chromium layer which is disrupted in the production process in such a way that it forms a nonconductive chromium oxide/chromium nitride layer with chromium particles embedded therein.

In a preferred embodiment, the conductivity detector zone is formed by a single-sided coating with nanoparticles on the topside or the bottom side of the spring element. Thus, preferably just one side of the spring element has a conductivity detector zone and a binding zone.

The main body of the spring element may comprise a wide variety of different materials and may comprise, for example, materials of low conductivity, for instance polymers, for example polyimide, carbon material or silicon-based material. Preferred silicon-based materials are silicon oxide, silicon carbide or silicon nitride. The material of the main body may also be a sandwich material.

The spring elements mentioned generally have the advantage that the electrical conductivity of the conductivity detector zone is dependent in a very sensitive manner on small changes in length. Such changes in length are caused, for example, by local contraction or expansion of the near-surface regions of the spring element. In particular, binding of molecules on the binding zone, on account of the resultant change in surface tension of the binding zone, can lead to bending of the spring element.

Binding molecules in the context of the present disclosure may be molecules that bind specifically to viral antigens, for example antibodies and antibody derivatives, antibody fragments such as single-chain antibodies, Fab fragments or (Fab)2 fragments. It is likewise possible to use alternative protein frameworks such as anticalins, lipocalins, receptors and fragments thereof, ankyrins, microbodies or aptamers.

In a preferred embodiment, the at least one binding molecule is an antibody or an antibody fragment. In the context of the present disclosure, the expression “at least one binding molecule” relates to at least one molecular species, for example an antibody species. Alternatively, it is also possible to use multiple different antibody species that bind to different antigens. In general, a large number of antibody molecules of one species are coupled to the binding zone.

In the context of the present disclosure, binding molecules that bind specifically to a viral antigen are binding molecules that bind to the antigen with an affinity (KD=k_{off}/kₒₙ) of at least KD=1×10⁻⁹ mol/l, more preferably at least 1×10⁻⁷ mol/l or at least 1×10⁻⁸ mol/l and most preferably of at least 1×10⁻⁹ mol/l. The binding of binder molecule and antigen can be determined, for example, by means of Biacore methods.

In a preferred embodiment, the antibody is a monoclonal, polyclonal or multiclonal, most preferably a monoclonal, antibody or an antibody fragment. The antibody is preferably an IgG antibody, but it is also possible to use other immunoglobulin classes. In addition, the antibody is preferably a recombinant antibody.

The proposed spring element may generally be designed for antigens of all viruses. In a particularly preferred aspect, the binding molecule binds specifically to an antigen of coronaviruses, especially an antigen of the SARS-CoV-2 virus. The antigen is preferably a peptide antigen, especially an antigen contained in the spike protein (S protein), envelope protein (E protein), membrane protein (M protein) or nucleocapsid protein (N protein) of the SARS-CoV-2 virus. In the context of the present disclosure, an antigen contained in a protein is defined by an amino acid sequence contained in the amino acid sequence of the protein in question. However, antigens in the context of the present disclosure may also be conformational antigens.

The antigen contained in the spike protein (S protein) may preferably have an amino acid sequence contained in one of the sequences identified by the GenBank accession numbers QII57161.1, QIC53213.1, QHR63290.2, QHR63280.2, QHR63270.2, QHR63260.2, QHR63250.2, YP_009724390.1 or QIA20044.1.

The antigen contained in the envelope protein (E protein) may preferably have an amino acid sequence contained in one of the sequences identified by the GenBank accession numbers QIA98556.1, BCA87373.1, BCA87363.1, QIM47478.1, QIM47469.1, QIM47459.1, QII87842.1, QII87832.1, QII87820.1, QII87808.1, QII87796.1, QII87784.1, QIK50450.1, QIK50440.1, QIK50429.1, QIE07483.1, QIE07473.1, QIE07463.1 or QIH55223.1.

The antigen contained in the nucleocapsid protein (N protein) may preferably have an amino acid sequence contained in one of the sequences identified by the GenBank accession numbers QIC53221.1, QII87776.1, QII87775.1, QHR63298.1, QHR63288.1, QHR63278.1, QHR63268.1, QHR63258.1, QHO62115.1 or QHO62110.1.

The antigen contained in the membrane protein (M protein) may preferably have an amino acid sequence contained in one of the sequences identified by the GenBank accession numbers QIC53216.1, QHR63293.1, QHR63283.1, QHR63273.1, QHR63263.1, QHR63253.1.

In a particularly preferred embodiment, the binding module binds to an antigen contained in the spike protein. The spike protein sticks out from the virus surface to a particular degree. Thus, antigens in the spike protein are of good steric accessibility for binding to a binding molecule, for example an antibody.

The binding molecules may be coupled directly to the material of the main body.

In a preferred embodiment, the main body is coated with a coating in the region of the binding zone. In this embodiment, the binding module is coupled to the coating, such that the coating is localized between the main body and the binding molecule.

The coating may comprise, for example, a precious metal, for instance Au or Pt. In addition, the coating may comprise nanoparticles.

The binding molecules may be coupled to the binding zone either covalently or non-covalently. The person skilled in the art is aware of various coupling methods for binding molecules such as antibodies (Jazayeri M H, Amani H, Pourfatollah A A, Pazoki-Toroudi H, Sedighimoghaddam B. Various methods of gold nanoparticles (GNPs) conjugation to antibodies. Sensing and Bio-Sensing Research. 2016; 9:17-22). In the case of an Au coating of the binding zone, for example, both for a covalent coupling and for a non-covalent coupling, in a first step, it is possible to pegylate onto the Au coating with a thiol-polyethylene glycol compound (thiol-PEG), for example with a thiol-polyethylene glycol acid or a thiol-polyethylene glycol ester. Covalent coupling of the antibody can then be effected, for example, by a known method by means of N-hydroxysuccinimide (NHS) and N-ethyl-N-[3-dimethylaminopropyl]carbodiimide (EDC).

In a preferred embodiment, the antibody is coupled to the Au coating by means of avidin/streptavidin binding. For this purpose, either streptavidin may be coupled covalently to the pegylated Au coating by means of N-hydroxysuccinimide (NHS) and N-ethyl-N-[3-dimethylaminopropyl]carbodiimide (EDC) in the aforementioned process, or thiol-PEG-biotin may be coupled directly to the Au coating in a first step and then streptavidin may be bound noncovalently to the Au-coupled thiol-PEG-biotin. In both cases, in the last step, there is noncovalent binding of a biotinylated antibody to the streptavidin coupled to the Au coating by means of the linkers mentioned.

In embodiments in which the binding zone is not coated, the person skilled in the art will be able to adapt the specified coupling methods for direct coupling to the material of the main body. In the case of silicon-based materials, the coupling can be effected, for example, on the basis of PEG silanes.

In a preferred embodiment, the biotinylated antibody is biotinylated in the Fc domain, preferably at the C-terminal end of the Fc domain. Corresponding biotinylation directs the antibody advantageously in such a way that the variable domains are directed away from the spring element in the direction of the surrounding medium and hence bind in a sterically unhindered manner to antigens in a medium in which viruses are to be detected.

The antigens bound to the binding molecules may be present in intact viruses, fragments of viruses or individual viral proteins.

The binding molecule, for example in an activated spring element, may comprise single-strand DNA (ssDNA) and/or other DNA fragments that bind specifically to DNA fragments in the sample. In an inert spring element, binding molecules may comprise single-strand DNA and/or other DNA fragments that do not bind to any chemical and/or biochemical and/or physical species in the sample, but match with the binding molecules of the activated spring element in characteristic parameters (e.g. chain length, chemical structure).

The binding molecule, for example in an activated spring element, may comprise single-strand RNA and/or other RNA fragments that bind specifically to RNA fragments in the sample. In an inert spring element, binding molecules may comprise single-strand RNA and/or other RNA fragments that do not bind to any chemical and/or biochemical and/or physical species in the sample, but match with the binding molecules of the active spring element in characteristic parameters (e.g. chain length, chemical structure).

The binding molecules, for example in an activated spring element, may comprise antibodies and/or other and/or further proteins that specifically bind target proteins. In an inert spring element, binding molecules may comprise specific isotype control antibodies and/or further proteins that do not bind to any chemical and/or biochemical and/or physical species in the sample.

The binding molecules may comprise scFv antibody components. An scFv antibody is a synthetically produced antibody fragment. By dividing an antibody into multiple fragments, it is possible to enhance the reactivity of the sensor to a low sample concentration.

The binding zone preferably comprises at least one hydrogel.

The nanoparticles of the conductivity detector zone are preferably metallic. More preferably, the nanoparticles are formed from chemically stable materials, most preferably from Au and/or Pt and/or Cr. The nanoparticles may preferably have an average particle size of up to 100 nm, more preferably up to 10 nm, provided that they are sufficiently electrically insulated from one another in the conductivity detector zone and their separations are sufficiently small that tunneling effects can be established between them.

The matrix of the conductivity detector zone is especially formed from organic, inorganic or dielectric material, for example from organometallic complexes, monomers, oligomers, polymers or mixtures of these monomers, oligomers and polymers. Processes for producing an above-described conductivity detector zone comprising nanoparticles embedded in a matrix are known to the person skilled in the art, for example from WO 2007/088018 A1.

The binding zone need not cover the entire surface of the spring element beyond the conductivity detector zone. Instead, the person skilled in the art may adjust and optimize the size and position of the binding zone depending on the signal generated by the conductivity detector zone.

The spring element may be configured such that a bond of an analyte and preferably of viral antigens to the binding molecules of the binding zone brings about a change in the surface tension of the binding zone.

The spring element may further be configured such that the change in the surface tension of the binding zone brings about bending of the spring element.

In addition, the spring element may consequently be configured such that binding of an analyte and preferably of viral antigens to the binding molecule in the binding zone brings about a change in the electrical conductivity in the conductivity detector zone. Thus, it is possible by the determination of the electrical conductivity of the conductivity detector zone to determine binding of analytes and preferably of antigens to binding molecules.

In one aspect, the spring element is configured such that binding of an analyte and preferably of viral antigens to the binding molecules in the binding zone brings about bending of the spring element.

Upon contacting of the proposed spring element with a medium containing viral antigens that is to be tested, it is thus possible via the variation in conductivity of the conductivity detector zone to determine the binding of viral antigens to the binding molecules in the binding zone of the spring element.

For this purpose, in a first method, it is possible to determine the conductivity of the conductivity detector zone prior to contacting of the spring element with a medium to be tested and after contacting of the spring element with a medium to be tested. By determining the change in conductivity, it is possible to infer and hence detect the presence of viral antigens in the medium to be tested.

In a further method, an above-described spring element comprising binding molecules that bind an analyte, preferably antigen, referred to hereinafter as activated spring element, and a corresponding spring element, but one that at least does not comprise any binding molecules as described above, referred to hereinafter as inert spring element, are used. In this method, both the activated spring element and the inert spring element are contacted with a medium to be tested. The activated and inert spring elements are configured such that the binding of analytes, preferably antigens, present in the medium to the binding molecules of the activated spring element in the conductivity detector zone of the activated spring element cause a greater change in conductivity than in the conductivity detector zone of the inert spring element by mere presence of the medium but without the specific binding of an analyte, preferably of antigens. By determining the difference in the conductivity of the conductivity detector zones of the activated and inert spring elements, it is possible to infer and hence detect the presence of the analyte, preferably of viral antigens, in the medium to be tested.

The spring elements thus enable, in a simple and rapid manner, the detection of an analyte, preferably of viral antigens, in a medium. The spring elements also have the advantage that these can be produced comparatively inexpensively in large numbers by means of established methods.

The spring elements may be used in various devices for detection of viruses.

Additionally proposed is a device for detecting an analyte, preferably viruses, comprising at least one above-described activated spring element, at least one electrical sensor for determining the conductivity of the conductivity detector zone of the spring element, and at least one comparator. The comparator may be configured such that it compares the actual conductivity value of a spring element that is in contact with a medium with a predetermined target value. The comparator is further configured such that it can infer the presence of the analyte and preferably of antigens in the medium from a variance between actual value and target value of the conductivity, and pass on a corresponding signal to an output device or a processor. In addition, the device comprises a power source for the electrical sensor, the comparator and optionally further elements.

A further device for detecting an analyte, preferably viruses, comprises at least one above-described activated spring element and at least one inert spring element, at least electrical sensors for determining the conductivity of the conductivity detector zones of the activated and inert spring elements, and at least one comparator. The comparator may be configured such that it compares the conductivity of the activated and inert spring elements that are in contact with a medium with one another. The comparator is further configured such that it can infer the presence of an analyte and preferably of antigens in the medium from a variance in the conductivity of the activated and inert spring elements, and pass on a corresponding signal to an output device or a processor. In addition, the device comprises a power source for the electrical sensor, the comparator and optionally further elements.

In a preferred embodiment, the activated and inert spring elements are interconnected in the form of a Wheatstone measurement bridge.

In a further aspect, a device for detecting the presence of an analyte in a sample, preferably for detecting the presence of viruses, in the form of a microfluidic chip is proposed, comprising a region configured for reception of a sample in a liquid medium, at least one microfluidic channel configured such that it guides the liquid medium into at least one measurement chamber, and at least one measurement chamber comprising at least one first and one second spring element. The first spring element is an activated spring element as described herein. The second spring element is an inert spring element as described herein. In addition, the device comprises electrical contacts connected to the conductivity detector zones of the first and second spring elements. The contacts are configured such that, when the chip is introduced into an evaluation device, they can be connected to corresponding contacts of the evaluation device.

The contacts of the evaluation device are connected to electrical sensors configured to determine the conductivity of the conductivity detector zones of the activated and inert spring elements of the microfluidic chip. In addition, the evaluation device comprises at least one comparator. The comparator may be configured such that it compares the conductivity of the activated and inert spring elements of the microfluidic chip that are in contact with a liquid medium with one another. The comparator is further configured such that it can infer the presence of an analyte, preferably of antigens, in the medium from a variance in the conductivity of the activated and inert spring elements, and pass on a corresponding signal to an output device or a processor.

The output device may be configured such that the presence of an analyte, preferably of viruses, in the medium tested is displayed as a binary yes/no. However, the output device may also output a signal proportional to the amount of analyte bound, preferably of antigens bound.

By means of corresponding coloring, the device may be configured so as to display a concentration of the analyte, preferably an antigen concentration, by means of the output device.

In addition, the evaluation device may comprise a power source for the electrical sensor, the comparator and optionally further elements.

In addition, the evaluation device may comprise a transmission unit, for example a radio transmission unit, by means of which data can be passed on from the processor to receiving devices. The evaluation device thus enables rapid distribution of relevant epidemiological data.

In a further aspect, a system for detecting an analyte, preferably viruses, comprising a microfluidic chip and an evaluation device is proposed.

The viruses in the various aspects are preferably coronaviruses, especially SARS-CoV-2.

Additionally proposed is a method of detecting viruses, comprising the contacting of a sample comprising an analyte, preferably a virus, with a spring element described. The sample is preferably a human or animal bodily fluid, for example saliva, blood, lymph, gastric juice, perspiration, a bodily excretion, for example urine or stool, or at least one cell. Cells and saliva as samples are preferably obtained in the form of a mucous membrane swab.

Depending on the type of sample, the contacting of the sample with the spring element may be direct or indirect. In general, the contacting is indirect, in that the sample is taken up, dissolved or suspended in an aforementioned medium and the liquid medium is contacted with the spring element as described above.

The spring element described herein or the devices described herein may be used in a method of diagnosing the infection of an individual with a virus. Thus, the devices described herein also relate to the use in a method of diagnosis of the infection of an individual with a virus. The method comprises at least the step of contacting a sample containing a bodily fluid or a cell from an individual with the spring element of the invention. The contacting with the spring element results in detection of the analyte and preferably of the virus as described above. It is thus possible to conclude an infection of the individual with the virus. In addition, the method may comprise the step of sampling.

By means of the devices described can small and mobile devices for rapid testing of viruses directly for medical personnel such as family doctors, paramedics or care personnel for provision, which can be used without any great prior knowledge. The proposed methods reacts very much more reliably at any time over the course of the infection compared to other test methods and leads to a clear electronic “YES” or “NO” message as to the presence of the virus in the sample tested. The results are available within a few minutes, and there is no need for time-consuming transport of the samples to the laboratory.

The technical scalability of the diagnosis platform enables the rapid testing of millions of people and hence real-time monitoring and anonymized control of the spread of the disease within the population. The connection of the evaluation device to the cloud allows new regional hotspots of virus spread to be recognized in real time and contained immediately. It is thus possible to use restrictions of freedom of movement and scarce health administration and hospital resources in a very much more targeted and efficient manner.

### BRIEF DESCRIPTION OF THE FIGURES

An exemplary embodiment is shown in the figure and is described in detail hereinafter.

FIG. 1(a) illustrates a spring element connected to an electrical sensor prior to binding of viruses to the spring element.

FIG. 1(b) illustrates a spring element connected to an electrical sensor after binding of viruses to the spring element.

### DETAILED DESCRIPTION OF EXEMPLARY EMBODIMENTS

In the following, a description of exemplary embodiments with reference to the figures are presented. Elements that are the same, similar or have the same effect are given identical reference numbers in the different figures, and repeated description of these elements is avoided to a certain degree in order to avoid redundancy.

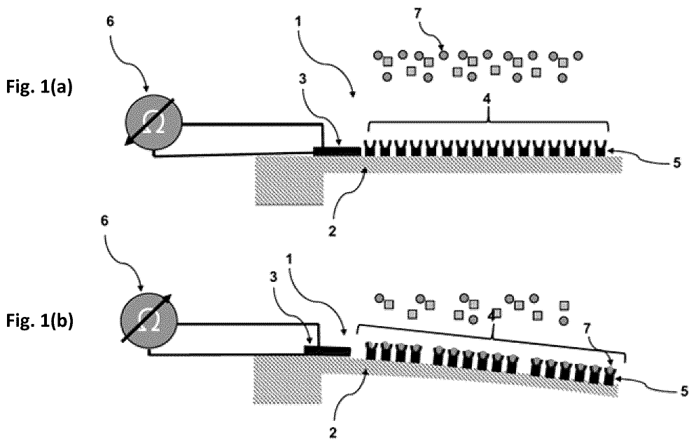

FIG. 1(a) shows a schematic diagram of the miniaturized spring element 1 with a flexible main body 2 having a conductivity detector zone 3 and a binding zone 4, wherein the electrical conductivity (σ) of the conductivity detector zone is determined by electronic tunneling, ionization or hopping processes, and wherein the conductivity detector zone is formed from nanoparticles embedded in a matrix that have higher electrical conductivity compared to the matrix material, and wherein the binding zone 4 comprises at least one binding molecule 5 that binds specifically to an analyte, preferably to viral antigens, and is coupled to the main body.

The conductivity detector zone is connected to an electrical sensor 6 for determining the conductivity of the conductivity detector zone 3.

The spring element 1 is configured such that binding of analytes, preferably of viral antigens 7, to the binding molecules 5 in the binding zone 4 brings about a change in the surface tension of the binding zone 4.

As shown in FIG. 1(b), the spring element 1 is configured such that the change in the surface tension of the binding zone brings about bending of the spring element 1. In addition, the spring element 1 is configured such that binding of an analyte, preferably of viral antigens 7, to the binding molecules 5 in the binding zone 4 brings about a change in the electrical conductivity of the conductivity detector zone 3. The conductivity of the conductivity detector zone 3 is determined with an electrical sensor 6. It is thus possible to determine binding of antigens 7 to binding molecules 5 by the determination of the electrical conductivity of the conductivity detector zone 3.

Although merely an illustrative embodiment has been disclosed in the preceding description, it is possible to undertake a wide variety of different changes and modifications thereto. The embodiment mentioned is merely an example and is not intended to restrict the scope of validity, applicability or configuration of the spring element in any way.

### LIST OF REFERENCE NUMERALS

1 spring element
2 main body
3 conductivity detector zone
4 binding zone
5 binding molecule
6 electrical sensor
7 viral antigens

1-24. (canceled)

25. A spring element for analyzing the presence of an analyte in a sample, comprising:
a flexible main body having a conductivity detector zone and a binding zone,
wherein a conductivity of the conductivity detector zone is determined by at least one of an electronic tunneling process, an ionization process and a hopping process,
wherein the conductivity detector zone comprises nanoparticles embedded in a matrix that have a higher electrical conductivity compared to a material of the matrix material, and
wherein the binding zone comprises at least one binding molecule that binds to the analyte and is coupled to the flexible main body.

26. The spring element as claimed in claim 25, wherein the binding module binds to viral antigens and is at least one antibody or antibody fragment.

27. The spring element as claimed in claim 25, wherein the binding module at least binds to viral antigens of coronaviruses.

28. The spring element as claimed in claim 27, wherein the viral antigens are antigens contained in a spike protein, an envelope protein, a membrane protein or a nucleocapsid protein.

29. The spring element as claimed in claim 26, wherein the at least one antibody is coupled to the main body by means at least one of avidin binding and streptavidin binding.

30. The spring element as claimed in claim 26, wherein the at least one antibody is an antibody biotinylated in the Fc domain.

31. The spring element as claimed in claim 25, wherein the at least one binding module is coupled covalently to the main body.

32. The spring element as claimed in claim 25, wherein the nanoparticles are metallic.

33. The spring element as claimed in claim 25, wherein the nanoparticles are at least one of Au nanoparticles, Pt nanoparticles and Cr nanoparticles.

34. The spring element as claimed in claim 25 wherein the matrix comprises an organic, inorganic or dielectric material.

35. The spring element as claimed in claim 25, wherein the spring element is configured such that binding of viral antigens to the binding module in the binding zone causes a change in the conductivity in the conductivity detector zone of the spring element.

36. The spring element as claimed in claim 25, wherein the spring element is configured such that binding of viral antigens to the binding module in the binding zone causes a change in a surface tension in the binding zone and the change in the surface tension in the binding zone causes a bending of the spring element.

37. The spring element as claimed in claim 25, wherein:
the binding molecule comprises at least one of a single-strand DNA (ssDNA) and other DNA fragments that bind to DNA fragments in a sample, or
in an inert spring element, binding molecules comprise at least one of single-strand DNA and other DNA fragments that do not bind to any chemical and/or biochemical and/or physical species in the sample, but match in characteristic parameters with the binding molecules of an activated spring element, or
the binding molecule comprises at least one of a single-strand RNA and other RNA fragments that bind to RNA fragments in the sample, or
in an inert spring element, binding molecules comprise at least one of a single-strand RNA and other RNA fragments that do not bind to any chemical and/or biochemical and/or physical species in the sample, but match in characteristic parameters with the binding molecules of an activated spring element, or
the binding molecule comprises at least one of antibodies and proteins that specifically bind target proteins, or
in an inert spring element, binding molecules comprise specific at least one of a isotype control antibodies and proteins that do not bind to any chemical and/or biochemical and/or physical species in the sample, or
the binding module comprises scFv antibody components.

38. A device for detecting the presence of an analyte in a sample comprising:
at least one spring element as claimed in claim 25;
an electrical sensor configured to determine the conductivity of the conductivity detector zone of the spring element; and
a comparator.

39. The device as claimed in claim 38, wherein the comparator is configured to compare an actual conductivity value of a spring element that is in contact with a medium with a predetermined target value, and further configured to infer a presence of the analyte in the medium from a variance between the actual value and a target value of the conductivity, and to transmit a corresponding signal to an output device.

40. A device for detecting an analyte, preferably viruses, comprising:
at least one activated spring element having at least one binding molecule that binds to the analyte as claimed in claim 25;
at least one inert spring element having no binding molecule that binds to the analyte;
electrical sensors configured to determine the conductivity of the conductivity detector zones of the activated and inert spring elements; and
at least one comparator.

41. The device as claimed in claim 40, wherein the comparator is configured to compare the conductivity of the activated and inert spring elements that are in contact with a medium with one another, and is further configured to infer a presence of the analyte in the medium from a variance in the conductivity of the activated and inert spring elements, and to transmit a corresponding signal to an output device.

42. The device as claimed in claim 41, wherein the activated and inert spring elements are interconnected in the form of a Wheatstone measurement bridge.

43. A device for detecting the presence of an analyte in a sample comprising:
a microfluidic chip having:
a region configured for reception of a liquid medium,
at least one microfluidic channel configured to guide the liquid medium into at least one measurement chamber,
at least one measurement chamber comprising at least one first spring element as claimed in claim 25 and a second spring element that does not comprise at least one binding molecule, and
electrical contacts connected to the conductivity detector zones of the first and second spring elements.

44. A method of detecting a presence of an analyte in a sample containing the analyte, the method comprising:
providing a flexible main body having a conductivity detector zone and a binding zone; and
determining a conductivity of the conductivity detector zone by at least one of an electronic tunneling process, an ionization process and a hopping process,
wherein the conductivity detector zone comprises nanoparticles embedded in a matrix that have a higher electrical conductivity compared to a material of the matrix material, and
wherein the binding zone comprises at least one binding molecule that binds to the analyte and is coupled to the flexible main body.

45. The method as claimed in claim 44, wherein the sample comprises at least one of a human or animal bodily fluid, bodily excretion or a cell.

46. A method of detecting the presence of an analyte having an activated spring element as claimed in claim 25 and having an inert spring element that does not comprise any binding molecules, wherein the activated and inert spring elements are configured such that the binding of analytes present in the medium to the binding molecules of the activated spring element in the conductivity detector zone of the activated spring element causes a greater change in the conductivity than in the conductivity detector zone of the inert spring element by a presence of the medium but without the binding of an analyte, preferably of antigens, the comprising:
contacting the activated spring element and the inert spring element with the medium to be tested;
determining a difference in conductivity of the conductivity detector zones of the activated and inert spring elements; and
determining the presence of the analyte in the medium to be tested.
